(19) United States
(12) Patent Application Publication
(10) Pub. No.: US 2017/0242042 A1
(43) Pub. Date: Aug. 24, 2017

(54) Presence of Phosphorylated Tau Protein in the Skin of Neurodegenerative Disease and SCIS, SCC, and BCC

(71) Applicants: Robert A. Norman, Tampa, FL (US); Maria Catano, San Luis Potosi (MX); Ildefonso Rodriguez-Leyva, San Luis (MX)

(72) Inventors: Robert A. Norman, Tampa, FL (US); Maria Catano, San Luis Potosi (MX); Ildefonso Rodriguez-Leyva, San Luis (MX)

(21) Appl. No.: 15/439,826

(22) Filed: Feb. 22, 2017

Related U.S. Application Data
(60) Provisional application No. 62/298,417, filed on Feb. 22, 2016.

(51) Int. Cl. G01N 33/68; G01N 33/574

(52) U.S. Cl. CPC G01N 33/6896; G01N 33/6893; G01N 33/5743; G01N 2800/202; G01N 2800/2814; G01N 2800/28

(57) ABSTRACT

The presence of misfolded proteins in the brain is the hallmark of neurodegenerative diseases. Protein aggregates could have systemic expression and might be found in several tissues including the skin. This demonstrates the presence of phosphorylated Tau (p-Tau) in the skin cells of patients with Alzheimer's Disease (AD). Antibodies against p-Tau (PHF, phosphorylated at S296 and AT8, phosphorylated at S202) were assayed in biopsied tissue from the retro-auricular area in 49 subjects: 20 with AD, 12 with nondegenerative dementia and 17 age-matched controls. Light and confocal microscopies were employed to localize Tau protein by immunohistochemistry and their presence in the skin was confirmed through Western blots. The skin biopsy taken from AD patients presented significantly higher levels of p-Tau (AT8: hyperphosphorylated at Ser 202) when compared both to control subjects and patients with non-degenerative dementia (p<0.001). This study demonstrates the presence of p-Tau in skin biopsies by immunoreactivity. This procedure could be used to support the clinical diagnosis of AD in living patients.

### FIELD OF INVENTION

This invention relates to the field of diagnostic methods for neurodegenerative disorders and cancer.

### BACKGROUND OF INVENTION

The increase in life expectancy of the population is associated with a higher incidence of neurodegenerative diseases. Their main feature is the expression of misfolded proteins [1-3]. The clinical differentiation among them is not always easy, since different proteinopathies can be expressed simultaneously. They are accompanied clinically by dementia, parkinsonism and/or motor dysfunction [4-9]. Currently, the definite diagnosis can only be done either demonstrating misfolded proteins in brain tissue or with genetic testing. Nevertheless, these proteins are also expressed outside the brain, not only in the peripheral nervous system but in the case of α-synuclein, also in other tissues such as the heart, gut and skin [10,11]. Tau is also expressed in skeletal muscle, lungs and kidneys. This opens the possibility of detecting proteinopathies in extra-cerebral tissues [12].

The brain and skin both share an ectodermal origin and therefore it is plausible to find molecular pathological alterations taking place in the skin as in the central nervous system. Since α-synuclein aggregates have been recently demonstrated in peripheral nerve terminals, epidermis and skin appendages of Parkinson's disease (PD) patients [13-15], our objective was to demonstrate the usefulness of demonstrating protein aggregates in the skin cells as a potential biomarker for AD living patients. For this purpose, phosphorylated Tau aggregates were analyzed by immunohistochemistry in the skin of patients with Alzheimer (AD) and non-degenerative dementia (NDD). These groups were compared with a control group with similar demographic characteristics.

### BRIEF DESCRIPTION OF THE DRAWINGS

For a fuller understanding of the invention, reference should be made to the following detailed description, taken in connection with the accompanying drawings, in which:

FIG. 1: Location of the five images obtained from each biopsy (A). Manual counting of immunopositive (green) and negative (red circles) cells (B). Image processing, only blue and green colors represent immunopositivity (C);

FIG. 2: Immunohistochemistry. Hippocampal neurons, PHF (A) and AT8 (B) with neurofibrillary tangles, in an AD patient, 100×, diaminobenzidine staining. Immunoreactivity patterns in epidermis of a control patient (C-D) and AD patient (E-F), with PFH and AT8 antibodies. 40×, amine-ethyl-carbazole staining;

FIG. 3: Immunohistochemistry patterns in skin appendages of an AD patient. AT8 antibody showed juxtanuclear staining in the pilosebaceous unit and eccrine glands. Amine-ethyl-carbazole staining. 40×;

FIG. 4: Skin immunofluorescence. Confocal microscopy. PHF (Cy5) and AT8 (Alexa Fluor 488) antibodies. Control subject (A-D) and AD patient (E-H). Cell nuclei stained with Sytox (C-G). Positive juxtanuclear staining is observed in AD patient with AT8 antibody. Scale bar 10 μm.

FIG. 5: Western Blot Demonstration of tau-protein in human epidermis. A) Epidermis of a control subject (PHF antibody) and B) Epidemis of AD patient (AT8 antibody);

FIG. 6: Immunopositivity for AT8 and PHF expressed as percentage of immunopositive pixels/total area. AD group AT8 expression had a p<0.001 as compared with the control group, likewise a p<0.001 as compared with the non-degenerative dementia group. Kruskal-Wallis followed by Mann-Whitney U-test were made for immunopositivity scores;

FIG. 7: demonstrates the results for Squamous Cell Carcinoma In Situ (SCIS);

FIG. 8: demonstrates the results for Squamous Cell Carcinoma (SCC); and

FIG. 9: demonstrates the results for Basal Cell Carcinoma (BCC).

### DETAILED DESCRIPTION OF THE PREFERRED EMBODIMENT

In the following detailed description of the preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention

Reagents and Antibodies

Reagents were analytical grade and the solutions were prepared in molecular grade water (18.2 O/cm). The following primary antibodies were employed: 1) rabbit monoclonal anti-tau (pSer396) [E178] (ab32057, Abcam, Cambridge, Mass.), which according to the supplier's instructions detects both phosphorylated and non-phosphorylated tau and 2) Mouse monoclonal anti-tau (pSer202+pThr205) [AT8] (MN1020, Thermo Scientific, Rockford, Ill.). A biotinylated secondary antibody was obtained from DAKO (Carpinteria, Calif.) and for immunofluorescence we employed a goat anti-mouse IgG antibody marked with Alexa Fluor 488 and a goat anti-rabbit IgG antibody marked with Cy5 (A1101 and A10523, Molecular Probes, Eugene, Oreg.). Cellular nuclei were evidenced with an orange nucleic acid stain, Sytox (S-11368, Molecular Probes, Eugene, Oreg.). Western blot reagents were molecular biology grade and obtained from IBI Scientific (Peosta, Iowa, USA); except for bis-acrylamide and molecular weight markers, which were obtained from BioRad (Hercules, Calif., USA).

Patient Selection

The patients were selected from the Neurology Department external practice in the Central Hospital in San Luis Potosi, Mexico, and all the participants or their tutors signed an informed consent letter. The Institutional Ethics and Research Committee previously authorized the protocol. The inclusion criteria were being diagnosed with AD, a non-neurodegenerative dementia or to be an apparently healthy asymptomatic subject who accepted to participate in this study. Three groups of patients were studied 1) Healthy subjects with similar age and demographic characteristics to the rest of the participants 2) Patients with a probable AD diagnosis according to the NINCDSARDA criteria [16] and 3) Subjects with a diagnosis of a nondegenerative dementia (NND) (normotensive hydrocephalous, subdural hematoma post-traumatic dementia, vascular dementia and epilepsy). The exclusion criteria were 1) patients with a local or systemic infectious process; 2) Severely impaired coagulation and 3) not accepting to be part of the study.

All AD and NDD subjects underwent cranial CT or MRI to aid their diagnosis; it was not a requisite for the control group.

Tissue Sampling and Processing

The participants were biopsied by a punch of 4 mm, taking a skin sample 3 cm behind the ear insertion, with previous local anesthesia and aseptic cleaning of the region, followed by compression for hemostasis. The biopsies were immersed in 0.1 M phosphate buffer containing 4% paraformaldehyde during 24 h and embedded in paraffin. Coronal 5 um sections were collected in electro-charged slides (Biocare Medical LLC, Concord, USA). Next, sections were dewaxed by heating (60° C., 10 min), followed by xylene and ethanol rinses, and rehydrated. For epitope recovery, slides with 3 tissue sections each were submerged in DIVA decloacker solution (Biocare Medical, LLC, Concord, Calif.) and placed in a pressure cooker during 30 min followed by a 20 min cooling time at room temperature. After 3 rinses with distilled water, endogenous peroxidase was depleted incubating the sections 15 minutes with 3% hydrogen peroxide. The sections then underwent incubation steps (15 min each) in a humidity chamber at room temperature to block unspecific background staining (Background sniper, Biocare Medical, LLC, Concord, Calif.) and endogenous biotin and biotin binding proteins (avidin/biotin blocking kit, Vector Laboratories Inc, Burlingame, Calif.) followed always by rinses with TBS-tween.

Monoclonal antibodies were incubated during 1 hour, followed by the streptavidin-biotin marked secondary antibody for 15 min. Peroxidase activity was visualized by incubating the sections with either diaminobenzidine, obtaining a brownish coloration, or with amine-ethylcarbazol to obtain a red coloration, and counterstained with Harris hematoxiline Negative controls consisted of tissue sections treated without the primary antibody. The sections were viewed with a Nikon microscope (NikonLabophot-2, Japan) equipped with a digital camera. For immunofluorescence, the primary antibodies were incubated overnight at 4° C., and analyzed with a confocal microscope (LEICA TCS SP2, Leica Microsystems GmbH, Heidelberg, Ger).

Western Blot

Fifty mg of rat brain or human skin were processed obtaining 150-500 μg of total protein by homogenization with a lysis buffer (0.025 M Tris, 0.15M NaCl, 0.001M EDTA, 1% NP-40 and 5% glycerol) at pH 7.4 and supplemented with a protease and phosphatase inhibitor cocktail (Santa Cruz Biotechnology, Inc., Santa Cruz, Calif.). The supernatant was recovered by centrifugation at 12000×g 10 min. Total protein was quantified by BCA assay. 50-80 μg were suspended in Laemli buffer 2× supplemented with 10% 13-mercaptoethanol, boiled 10 min at 95° C. and run on SDS-PAGE 10-15% gel. The proteins were electrophoretically transferred to 0.2 PVDF membranes (BioRad Laboratories, Hercules, Calif.) and blocked with 3% bovine serum albumin (BSA) in Tris-buffered saline (pH 7.6) containing 0.1% Tween (TBST) and probed overnight at 4° C. with PHF-Tau (1:500) and AT8 (1:250). In addition, all membranes were probed with an antibody directed against β-actin as a control protein in whole lysates. After washing with TBST, the membranes were incubated with secondary antibodies HRP-conjugated (1:5000) in TBST+3% BSA during 1 h 30 min After subsequent washes, bands were visualized by chemiluminescence (Pierce, Rockford, Ill. USA) followed by autoradiography.

Evaluation and Statistical Analysis

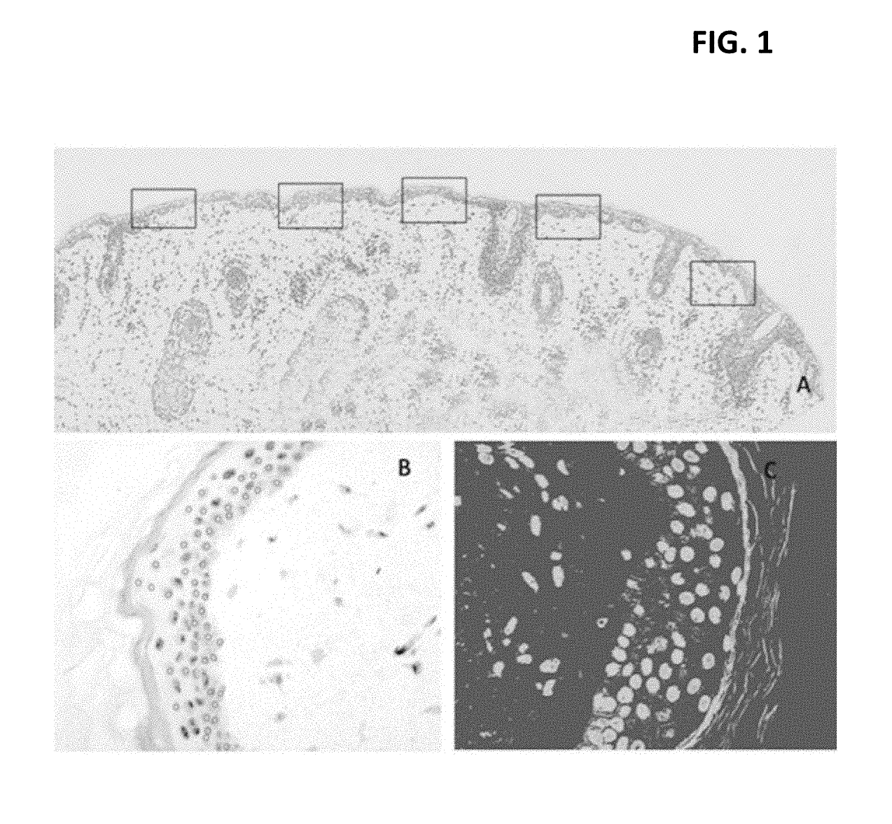

The conformation of the three groups was made with the clinical presentation, MMSE, laboratory and image features. The pathologist was blind about the clinical diagnosis. In order to validate the evaluation procedure, 5 fields per section were captured at the same magnification (40×) and the immunopositive and immunonegative cells were manually counted using the Photoshop software (V 12.1 Adobe Systems Inc, San Jose Calif.) in the obtained color images; this quantification was finally expressed in percentage for each one of the participants (FIG. 1A).

TABLE 1
Significant differences among groups were found in MMSE scores and % immunopositivity
for AT8. A p < 0.001 as compared with the control group, B p < 0.001 as
compared with the AD group. ANOVA followed by Tuckey test for the variables age and
MMSE. Kruskal-Wallis followed by Mann-Whitney U-test for immunopositivity percentages.

| | | | Age | MMSE | Immunopositivity percentage | |
|---|---|---|---|---|---|---|
| | Number of | Male/ | (Years, | (Points, | (median, min-max) | |
| Condition | Subjects | Female | Mean ± SD) | Mean ± SD) | PHF | AT8 |
| Healthy | 17 | 6/11 | 72 ± 14 | 29.4 ± 1.4 | 36 (9-58) | 0.8 (0.4-1.7) |
| Alzheimer's | 20 | 6/12 | 79 ± 9 | 14.5 ± 7.3^{A} | 45 (0-67) | 4.6 (2.7-5.4)^{A} |
| disease | | | | | | |
| Non-degenerative | 12 | 9/3 | 76 ± 13 | 17.2 ± 9.7^{A} | 46 (15-88) | 1.4 (0.3-1.7)^{B} |
| dementia | | | | | | |

The quantification of immunopositivity, estimated by manual cell counting performed by two separated observers was compared, obtaining a 95% of correlation (FIG. 1B). To corroborate these estimates and to obtain a confident and unbiased evaluation we also used a digital image processing. We employed the numerical principles from the model RGB (red, green, blue); 5 images from each sample of skin were evaluated, quantifying the total area and the expression of immunopositivity (FIG. 1C). The images were processed by means of the commercial software Image Pro Plus 7 (MediaCybernetics, Bethesda, Md., USA). The kappa coefficient between the two employed methods was 0.8.

The analyzed structure was the epidermis. A parametrical statistical test was applied in order to compare age and MMSE among the 3 analyzed groups, considering values of p less than 0.05 as being significant. Percentages of immunopositivity were subjected to a nonparametric analysis by means of Kruskal-Wallis test followed by UMann Whitney to make the comparison between groups.

Results

Control subjects and patient were in the same age range (p=0.7). The control group was confirmed by 17 subjects without neurological disease (normal MMSE); 20 patients with AD (mean MMSE 14) constituted the second group and individuals that presented nondegenerative dementias (mean MMSE 17) constituted the third group of 12 subjects (Table 1). Significant differences among groups were found in MMSE scores between AD and control (p<0.001) and the NDD and control group (p<0.001).

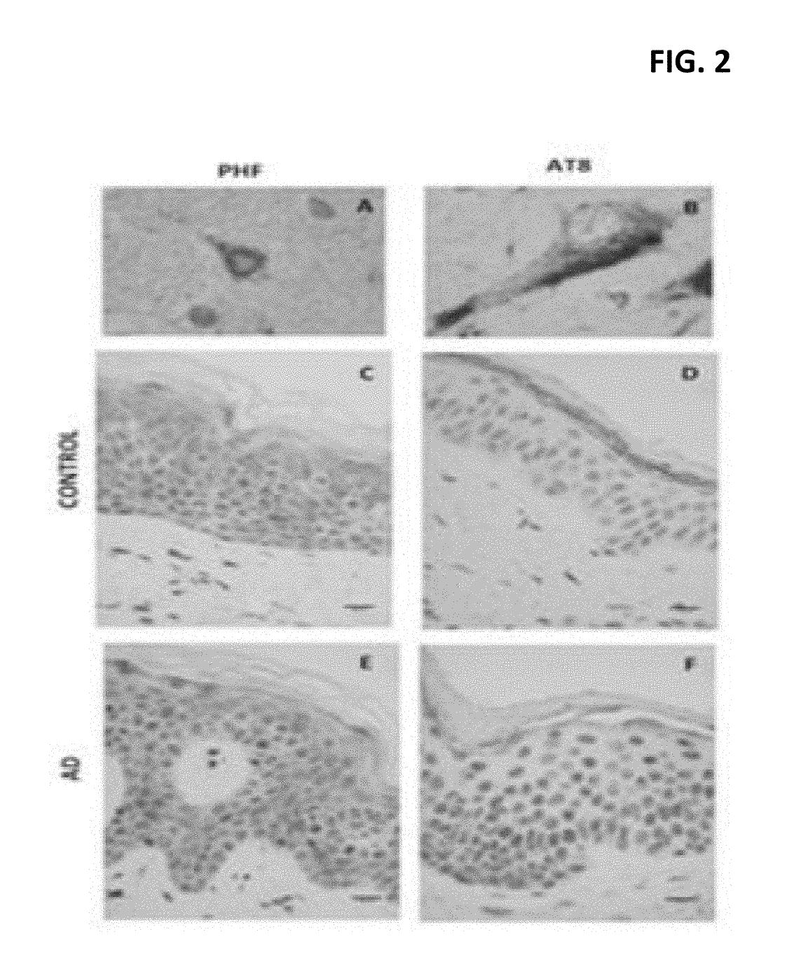

The employed antibodies were previously tested in autopsied brain tissue from a confirmed AD cadaver. Both anti-tau antibodies reacted positively with cortical and hippocampal neurons presenting neurofibrillary tangles (FIGS. 2A and 2B). Both Anti-AT8 and anti-PHF antibodies showed immunoreactivity localized to the cytoplasm of the affected neurons, and in the neuropil of the cortical and hippocampal regions. Next, the immunoreactivity pattern in control skin biopsies was analyzed. Abundant cytoplasmic positivity and scarce positive nuclear staining was observed in epidermal cells with PHF antibody (FIG. 2C), nerve twigs were also stained. Null to scarce immunopositivity was detected to the AT8 epitope in control subjects (FIG. 2D). In the skin from AD (FIG. 2E) a clear PHFpositivity was demonstrated.

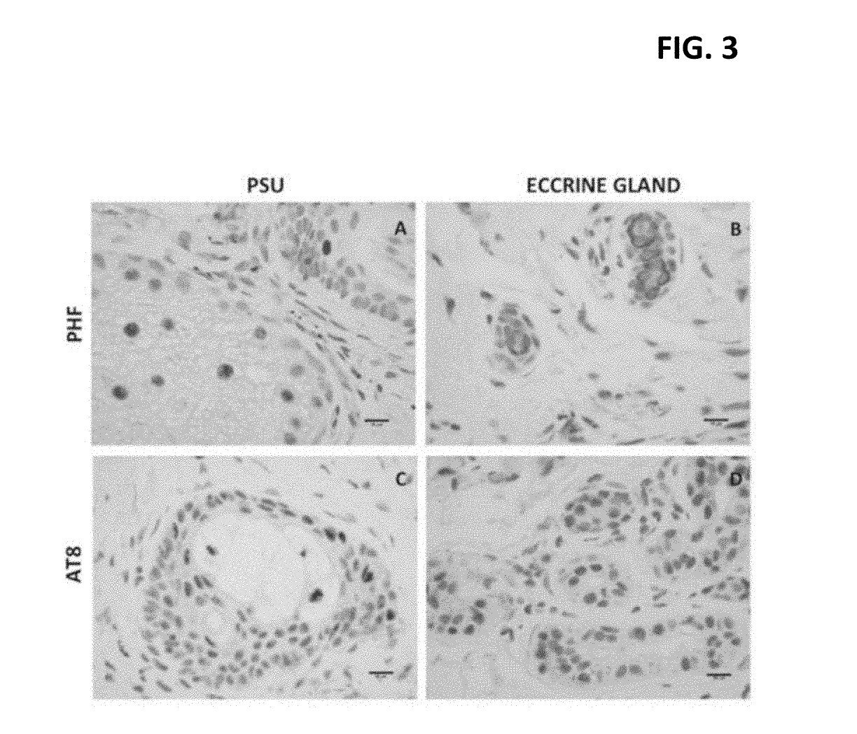

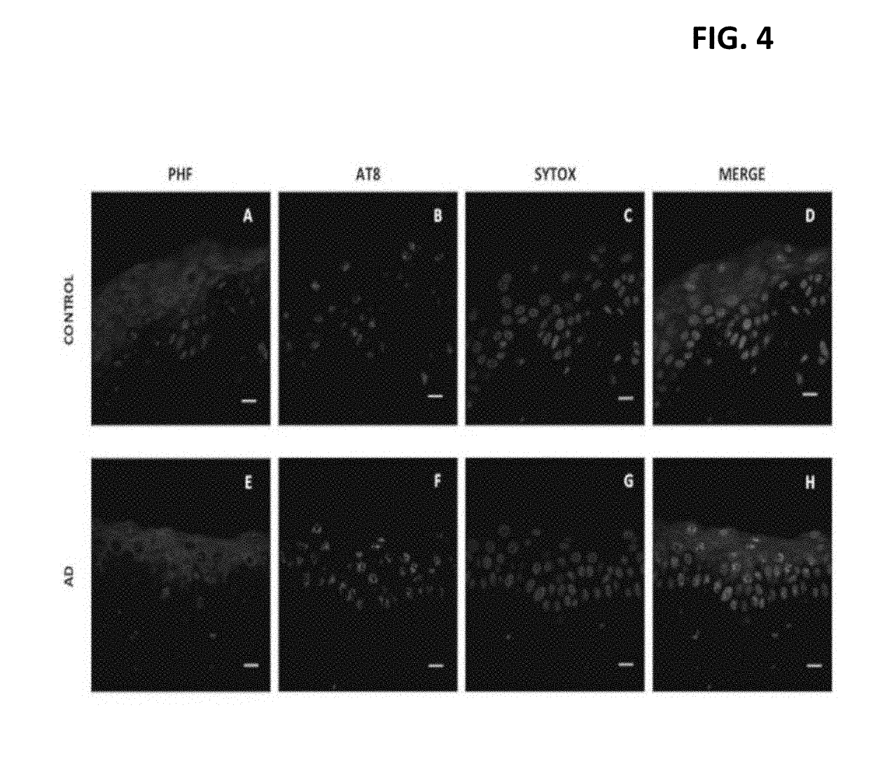

Also, in AD patients (FIG. 2F) we found juxtanuclear red expression, with granular staining involving keratinocyte nuclei throughout the thickness of the epidermis with AT8 antibody. When the skin appendages were evaluated (FIG. 3), the pilosebaceous unit (PSU) of AD patients showed a strong red juxtanuclear immunopositivity to PHF and AT8 in both, PSU (A,C) and eccrine gland (B,D). PHF antibody stained the cytoplasm of eccrine gland cells intensely, while cell nuclei were immunopositive to AT8 [17]. The nuclear and juxtanuclear localization of abundant AT8 immunopositivity in AD patients was confirmed by immunofluorescence (FIG. 4F), while the PHF epitope showed immunopositivity both in control (4A) and AD patients (4E), with a predominant cytoplasmic localization (FIGS. 4A and 4E).

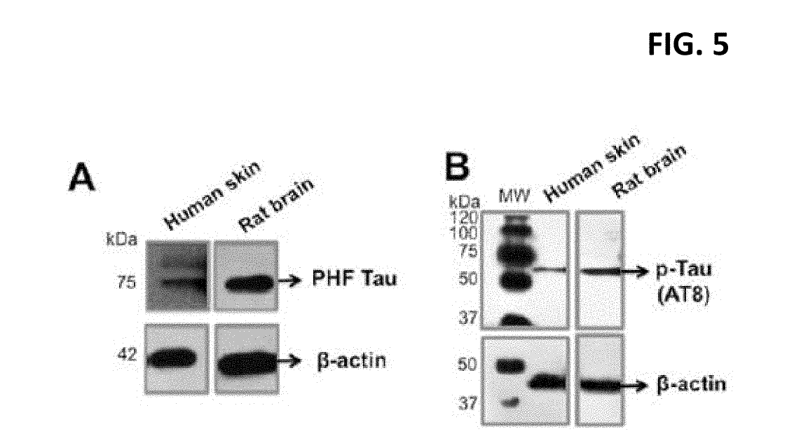

The localization of the granules was confirmed with the staining to delineate nuclei in red (Sytox, FIGS. 4C and 4G) and by merging the images from both antibodies. A clearly different pattern of immunofluorescence between controls and patients is observed in these images, marked by a difference in AT8 immunostaining (FIGS. 4D and 4H). The comparative analysis of the control group with AD patients showed that controls presented sparse, green, juxtanuclear granules in few keratinocytes with AT8 antibody (FIG. 4B); versus frequent juxtanuclear green granules in AD pateints (FIG. 4F). The presence of positivity to AT8 in keratinocytes was clear in the merged image (FIG. 4H). The identity of p-Tau in human skin was confirmed through Western blots (FIG. 5).

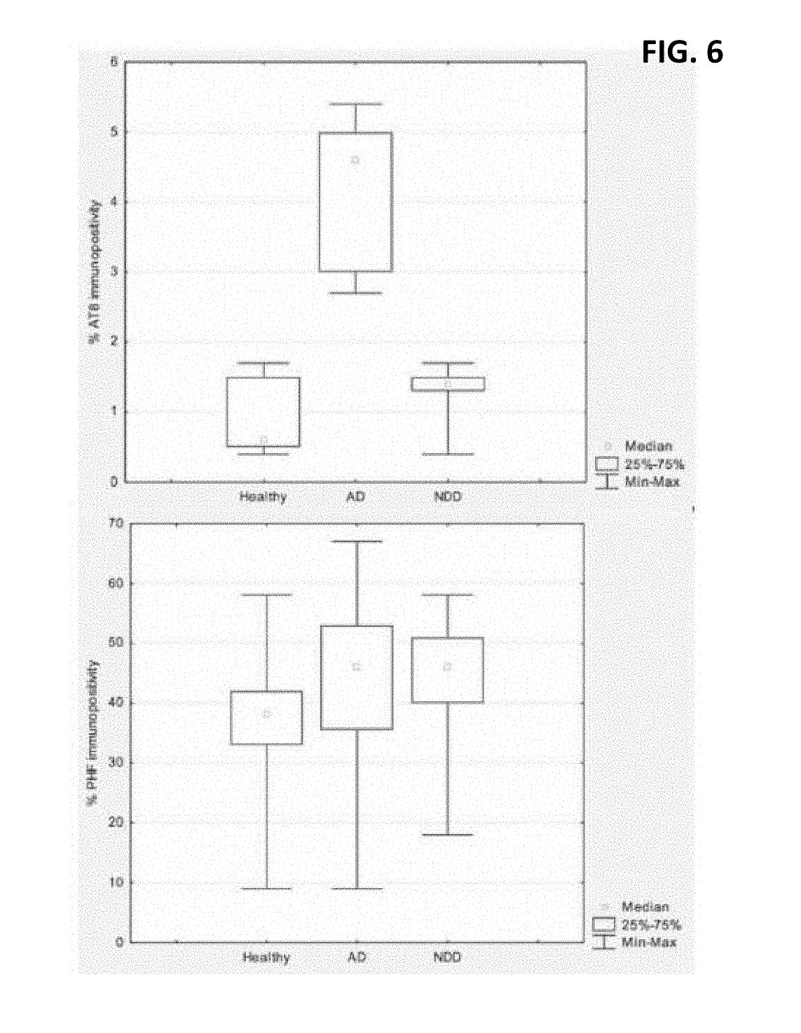

Although protein aggregates were detected in the different epidermis strata and skin appendages, the structure that allowed an in depth analysis and comparison of protein immunoreactivity was the epidermis (FIG. 6) Immunoreactivity to PHF antibodies was similar in the 3 groups of patients showing not statistically significant difference [H(2, N=49)=5.1, p=0.08]. By contrast, immunoreactivity to AT8 antibodies was significantly different among groups [H(2, N=49)=35.8, p<0.001]. The analysis between groups showed that AD patients presented significantly higher AT8 immunoreactivity than controls and patients suffering from non-degenerative dementias (p<0.001).

Discussion

A skin biopsy was taken from a sample of 49 subjects with similar demographic characteristics. When we compared the group of AD, this presented significantly higher levels of p-Tau (AT8:hyperphosphorylated at Ser 202) than both control and nondegenerative dementia group. These antibodies were chosen because in human studies, Ser396 phosphorylation (PHF] has been reported as an event that starts early in neurodegeneration and increases with aging and with the disease progress. Phosphorylation at Ser202 on the other hand is always present and significantly increased in diseased brains when compared to controls [18].

These phosphorylation sites have also been reported in the reversible tau hyperphosphorylation that takes place in hibernating animals proposed as a model for the study of taupathies. In this model, both sites (S396 and S202) present an important increase in phosphorylation during that physiological period [18,19]. We confirm here by means of Western blot the presence of p-Tau in the skin showing a similar molecular weight as in other organs [20-22]. The presence of p-Tau in the skin provides thus relevant information, and in our model AT8 (S202) immunoreactivity was significantly associated with the presence of AD.

The skin opens interesting possibilities for the study of neurodegenerative diseases because it is highly innervated and has the capacity of producing and releasing several neuropeptides [23]. It also expresses genes involved in neurological diseases like APP, Tau, PSEN1 and PARK2 among others [24]. According to this, several diseases from the nervous system have dermal manifestations, as it is the case of PD, where there is an increased risk of melanoma and patients present seborrhea and hyperhidrosis besides the classical motor manifestations of the disease [25].

In addition to becoming aggregated, Tau and other proteins that participate in neurodegenerative diseases translocate inside the cell nucleus [26,27] and interact with DNA [28, 29]. Current evidence supports that Tau participates in stress response protecting DNA [30,31], promotes chromosome stability by means of its interaction with both microtubules and chromatin [32], and upon DNA binding allows expression of silent genes immersed in heterochromatin [33]. These epigenetic changes however, are regarded as a source of neurodegeneration promoted by Tau, since it leads to the expression of aberrant genes in neurons. It is then of great interest that skin biopsies provide the possibility to explore Tau behavior in relation with epigenetic modifications.

Biomarkers of neurodegenerative diseases have been developed in recent years, mainly based on advanced molecular neuroimaging. Most health systems in the world, however, cannot afford these studies for the clinical practice. Therefore, skin biopsies represent an alternative to support the diagnosis of the two most important neurodegenerative diseases, by applying immunohistochemistry with commercially available antibodies. This can be done in a standard pathology lab of worldwide hospitals and clinical laboratories.

Comparison of Phosphorylated Tau and Ki-67 Staining in SCIS, SCC, and BCC

While Tau is a well-known component in the pathogenesis of Alzheimer disease, it normally functions to bind microtubules during cell division in healthy cells. Expression of phosphorylated tau has been demonstrated in lung, breast, prostate, and bladder carcinomas, however, it has not been studied in non-melanoma skin cancers.

We identified skin cancers from routine specimens submitted for pathology analysis from nursing home patients. Squamous cell carcinoma in situ (SCIS), Invasive squamous cell carcinoma (SCC,) and Basal cell carcinoma (BCC) samples were acquired via shave biopsies from the nose, right ear, and right cheek, respectively. The samples were stained with rabbit monoclonal Tau antibodies directed to different phosphorylation sites (Serine 202, Serine 396, and Threonine 231) and with Ki-67 rabbit antibodies. Tau immunopositivity was remarkable with anti-phosphoSerine202 (p-Tau).

The SCIS showed p-Tau immunopositivity within the epidermis (cytoplasmic and nuclear), whereas SCC staining was found predominantly in the periphery of the tumor (cytoplasmic and nuclear). In BCC, the p-Tau staining was in both the basal center and periphery of the tumor (cytoplasmic predominately). Ki-67 nuclear staining was more extensive than that of p-Tau. SCIS had the most Ki-67 staining at the base of the tumor, while SCC had predominantly peripheral staining. BCC also stained positive for Ki-67 mostly at the periphery of the tumor, with some additional staining in the tumor center.

Ki-67 is a well-known proliferation marker, and appears to be a more sensitive marker than p-Tau in non-melanoma skin cancers. Previous studies have also established that high expression phosphorylated Tau was associated with breast cancers that were more chemotherapy resistant. Whether phosphorylated tau in skin cancers translates into a more aggressive behavior remains to be explored in future studies. The significance of cytoplasmic vs nuclear staining remains to be defined. The utility of p-Tau staining may find an application in therapies that target mitotic activity, such as superficial radiation.

Tau and Ki-67 are well known proteins which are involved in cell division. Tau, a structural protein, is critical in giving stability to microtubules which physically separate the chromosomes during mitosis. Numerous carcinomas have been studied with regards to Tau, including breast³⁴,³⁵, bladder³⁶, gastric³⁷, and ovarian³⁸ cancer. While non-melanoma skin cancer survivors may have lower rates of Alzheimer's Disease when compared to people without a history of non-melanoma skin cancer³⁹, the explicit presence of Tau has not been studied in skin cancer, specifically Basal Cell Carcinoma (BCC), Squamous Cell Carcinoma (SCC), and Squamous Cell Carcinoma In Situ (SCIS). Ki-67, a protein with non-histone characteristics⁴⁰, is involved strictly in cell proliferation⁴¹. Previous studies have shown that Ki-67 expression increases with advancement of cervical intraepithelial neoplasia and squamous cell carcinoma⁴², with its expression being associated with the malignancy of the tumor⁴⁰.

Objectives: to investigate P-Tau and Ki-67 staining in BCC; to investigate P-Tau and Ki-67 staining in SCC; to investigate P-Tau and Ki-67 staining in SCIS; to compare P-Tau and Ki-67 staining patterns in BCC, SCC, and SCIS; and to compare the P-Tau staining pattern in BCC, SCC, and SCIS to those found in other carcinomas.

Methods

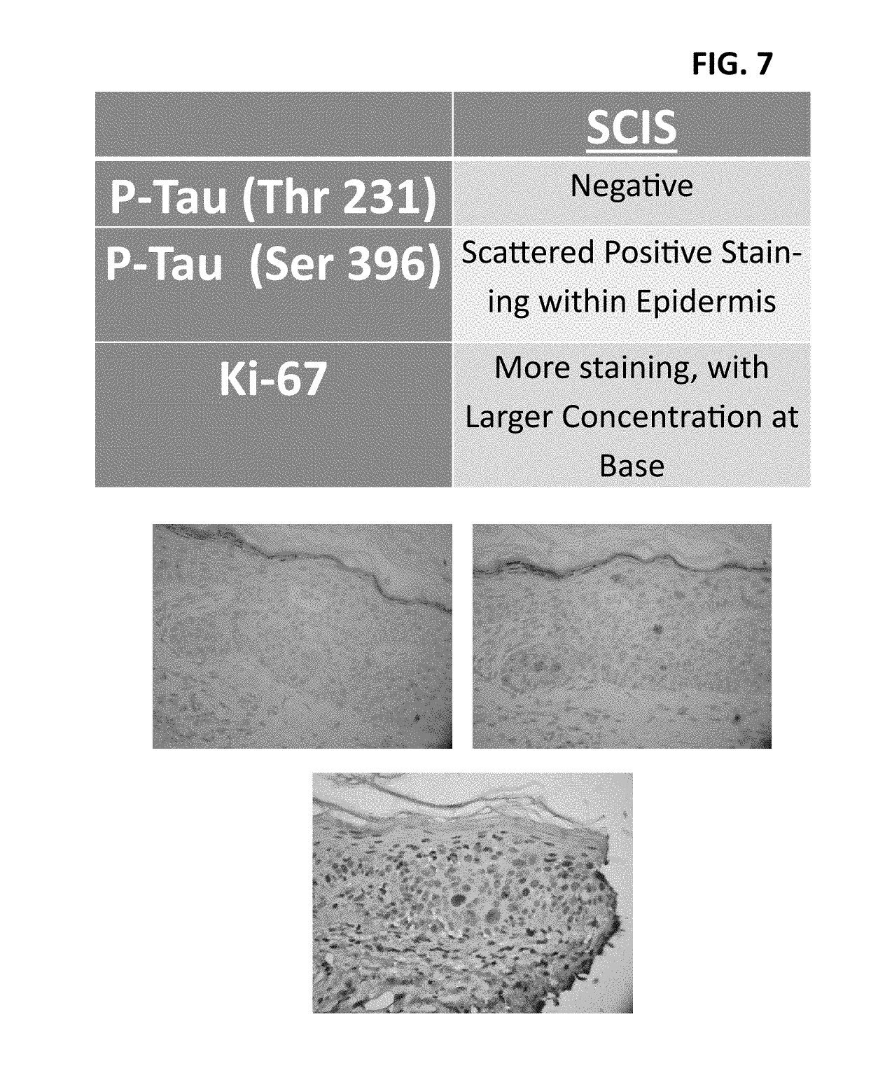

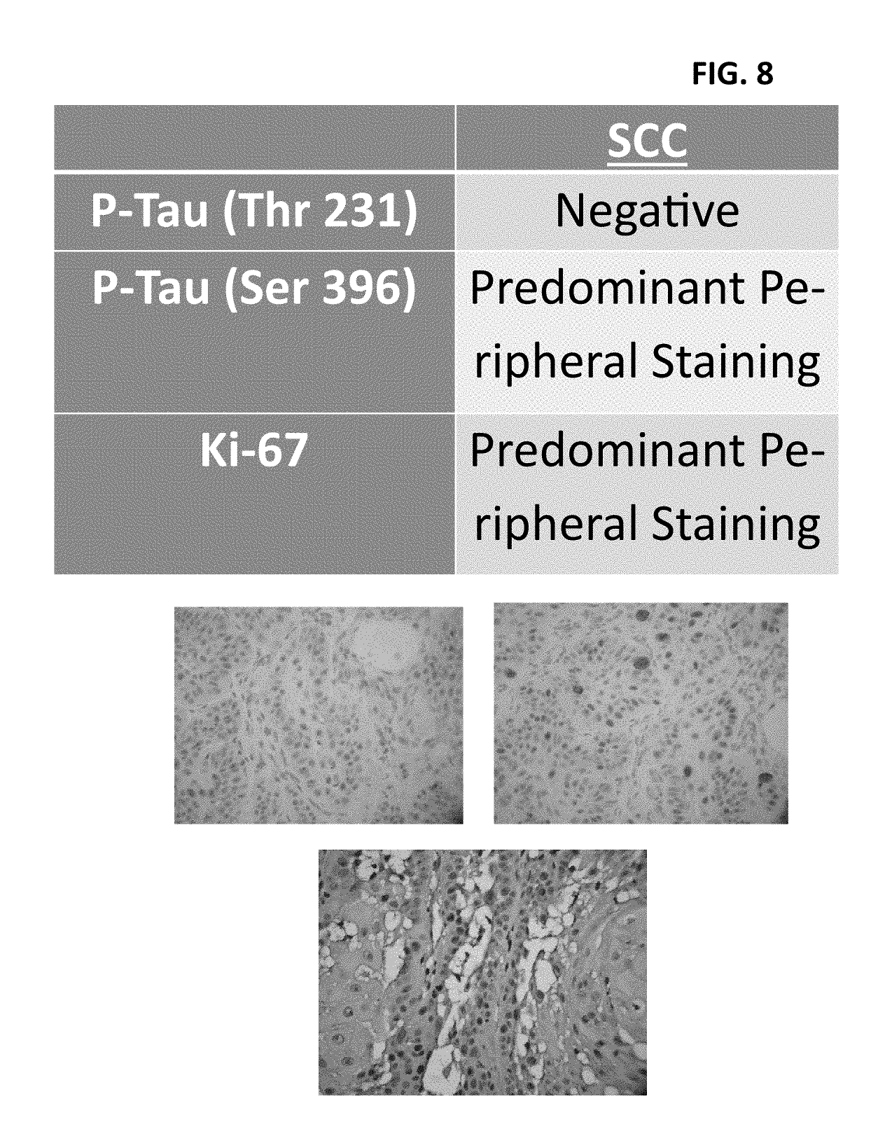

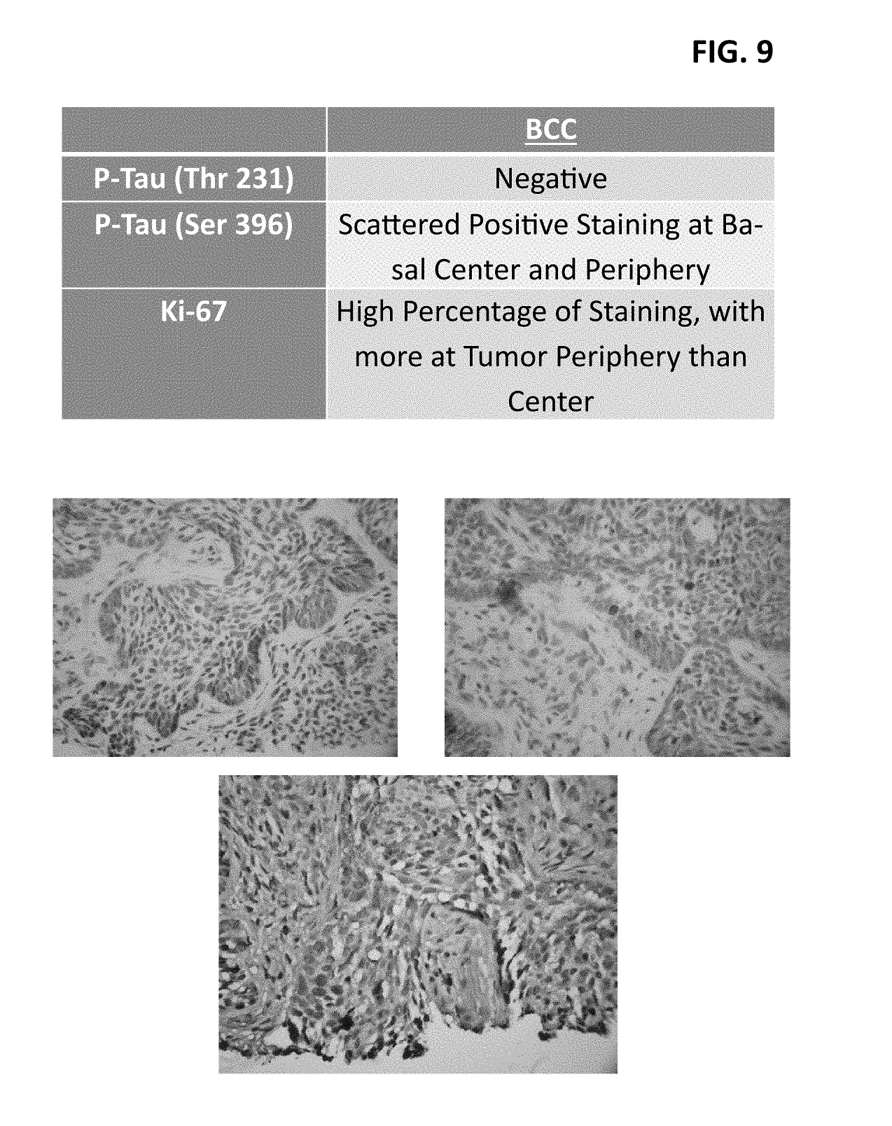

Skin cancers were identified from routine specimens submitted for pathology analysis from nursing home patients. Our SCIS, SCC, and BCC samples were acquired via shave biopsies from the nose, right ear, and right cheek, respectively. The samples were stained with rabbit monoclonal Tau antibodies directed to different phosphorylation sites (Serine 396, and Threonine 231) and with Ki-67 rabbit antibodies. Results are shown in FIGS. 7-9.

To our knowledge, our study is the first to show P-Tau in skin cancer cells, with Ki-67 staining more prominently. Ki-67 is present during the active phases of the cell cycle⁴¹, so a cancer cell which is constantly dividing will stain positively. Ki-67 has been shown to be positively related to the advancement of cervical intraepithelial neoplasia and squamous cell carcinoma⁴². Smith et al postulated a role of Ki-67 in producing abnormally phosphorylated Tau protein⁴³, which may explain why Ki-67 stained more prominently in our study.

Two studies have also established that high expression of p-Tau was associated with breast cancers that were more chemotherapy resistant. While Tau has been used as a marker for neurological diseases, whether phosphorylated tau in skin cancers translates into a more aggressive behavior remains to be explored in future studies, as does the significance of cytoplasmic vs nuclear staining. The utility of p-Tau staining may find an application in therapies that target mitotic activity, such as superficial radiation.

References: The following materials, to which reference is made above, are incorporated by reference as though fully contained herein:

1. Iqbal K, Wang X, Blanchard J, Liu F, Gong C X, et al. (2010) Alzheimer's disease neurofibrillary degeneration: pivotal and multifactorial. Biochem Soc Trans 38: 962-966.

2. Lee S J, Lim H S, Masliah E, Lee H J (2011) Protein aggregate spreading inneurodegenerative diseases: problems and perspectives. Neurosci Res 70: 339-348.

3. Gotz J, Matamales M, Gotz N N, Ittner L M, Eckert A (2012) Alzheimer's disease models and functional genomics-How many needles are there in the haystack? Front Physiol 3: 320.

4. Huang Y, Mucke L (2012) Alzheimer mechanisms and therapeutic strategies. Cell 148: 1204-1222.

5. Trojanowski J Q, Lee V M (1998) Aggregation of neurofilament and alpha-synuclein proteins in Lewy bodies: implications for the pathogenesis of Parkinson disease and Lewy body dementia. Arch Neurol 55: 151-152.

6. Sampathu D M, Neumann M, Kwong L K, Chou T T, Micsenyi M, et al.(2006) Pathological heterogeneity of frontotemporal lobar degeneration with ubiquitin-positive inclusions delineated by ubiquitin immunohistochemistry and novel monoclonal antibodies. Am J Pathol 169: 1343 1352.

7. Pouclet H, Lebouvier T, Coron E, Rouaud T, Flamant M et al. (2012) Analysis of colonic alpha-synucleinpathology in multiple system atrophy. Parkinsonism Relat Disord 18: 893-895.

8. Mollenhauer B, Locascio J J, Schulz-Schaeffer W, Sixel-Doring F, Trenkwalder C et al. (2011) a-synuclein and tau concentrations in cerebrospinal fluid of patients presenting with parkinsonism: a cohort study. Lancet Neurol 10: 230-240.

9. Neumann M, Kwong L K, Sampathu D M, Trojanowski J Q, Lee V M (2007) TDP-43 proteinopathy in frontotemporal lobar degeneration and amyotrophic lateralsclerosis: protein misfolding diseases without amyloidosis. Arch Neurol 64: 1388-1394.

10. Beach T G, Adler C H, Sue L I, Vedders L, Lue L, et al. (2010) Multi-organ distribution of phosphorylatedalpha-synuclein histopathology in subjects with Lewy body disorders. Acta Neuropathol 119: 689-702.

11. Lebouvier T, Tasselli M, Paillusson S, Pouclet H, Neunlist M, et al. (2010) Biopsable neural tissues: toward new biomarkers for Parkinson's disease? Front Psychiatry 1: 128. 12. Ramachandran G, Udgaonkar J B (2013) Mechanistic studies unravel the complexity inherent in tau aggregation leading to Alzheimer's disease and the taupathies. Biochemistry 52: 4107-4126.

13. Rodriguez-Leyva I, Calderon-Garcidueñas A L, Jimenez-Capdeville M E, Rentería-Palomo A, Hernandez-Rodriguez H G, et al. (2014) a-synuclein inclusions in the skin of Parkinson's disease and parkinsonism. Ann ClinTransl Neurol 1: 471-478.

14. Wang N, Gibbons C H, Lafo J, Freeman R (2013) a-synuclein in cutaneous autonomic nerves. Neurology 81: 1604-1610.

15. Doppler K, Ebert S, Uceyler N, Trenkwalder C, Ebentheuer J, et al. (2014) Cutaneous neuropathy in Parkinson's disease: a window into brain pathology. Acta Neuropathol 128: 99-109.

16. Mirra S S, Heyman A, McKeel D, Sumi S M, Crain B J, et al. (1991) The Consortium to Establish a Registry for Alzheimer's Disease (CERAD). Part II. Standardization of the neuropathologic assessment of Alzheimer's disease. Neurology 41: 479-486.

17. Daniel S E, Lees A J (1993) Parkinson's Disease Society Brain Bank, London: overview and research. J Neural Transm Suppl 39: 165-172.

18. Mondragon-Rodriguez S, Perry G, Luna-Munoz J, Acevedo-Aquino M C, Williams S (2014) Phosphorylation of tau protein at sites Ser(396-404) is one of the earliest events in Alzheimer's disease and Down syndrome. Neuropathol Appl Neurobiol 40: 121-135.

19. Arendt T, Stieler J, Strijkstra A M, Hut R A, Rüdiger J, et al. (2003) Reversible paired helical filament-like phosphorylation of tau is an adaptive process associated with neuronal plasticity in hibernating animals. J Neurosci 23: 6972-6981.

20. Stieler J T, Bullmann T, Kohl F, Toien Ø, Bruckner M K, et al. (2011) The physiological link between metabolic rate depression and tau phosphorylation in mammalian hibernation. PLoS One 6: e14530.

21. Goedert M, Jakes R (1990) Expression of separate isoforms of human tau protein: correlation with the tau pattern in brain and effects on tubulin polymerization. EMBO J 9: 4225-4230.

22. Bullmann T, Holzer M, Mori H, Arendt T (2009) Pattern of tau isoforms expression during development in vivo. Int J Dev Neurosci 27: 591-597.

23. Clos A L, Kayed R, Lasagna-Reeves C A (2012) Association of skin with the pathogenesis and treatment of neurodegenerative amyloidosis. Front Neurol 3: 5.

24. Makrantonaki E, Brink T C, Zampeli V, Elewa R M, Mlody B, et al. (2012) Identification of biomarkers of human skin ageing in both genders. Wnt signalling—a label of skin ageing? PLoS One 7: e50393.

25. Antonini A, Barone P, Marconi R, Morgante L, Zappulla S, et al. (2012) The progression of non-motor symptoms in Parkinson's disease and their contribution to motor disability and quality of life. J Neurol 259: 2621-2631.

26. Lu J, Li T, He R, Bartlett P F, Gotz J (2014) Visualizing the microtubuleassociated protein tau in the nucleus. Sci China Life Sci 57: 422-431.

27. Zhou M, Xu S, Mi J, Ueda K, Chan P (2013) Nuclear translocation of α-synuclein increases susceptibility of MES23.5 cells to oxidative stress. Brain Res 1500: 19-27.

28. Camero S, Benitez M J, Jimenez J S (2013) Anomalous protein-DNA interactions behind neurological disorders. Adv Protein Chem Struct Biol 91: 37-63.

29. Sjoberg M K, Shestakova E, Mansuroglu Z, Maccioni R B, Bonnefoy E (2006) Tau protein binds to pericentromeric DNA: a putative role for nuclear tau in nucleolar organization. J Cell Sci 119: 2025-2034.

30. Sultan A, Nesslany F, Violet M, Begard S, Loyens A, et al. (2011) Nuclear tau, a key player in neuronal DNAprotection. J Biol Chem 286: 4566-4575.

31. Camero S, Benítez M J, Barrantes A, Ayuso J M, Cuadros R (2014) Tau protein provides DNA with thermodynamic and structural features which are similar to those found in histone DNA complex. J Alzheimers Dis 39: 649-660.

32. Rossi G, Dalpra L, Crosti F, Lissoni S, Sciacca F L (2008) A new function of microtubule-associated protein tau: involvement in chromosome stability. Cell Cycle 7: 1788-1794.

33. Frost B, Hemberg M, Lewis J, Feany M B (2014) Tau promotes neurodegeneration through global chromatin relaxation. Nat Neurosci 17: 357-366.

34. Zhou J, Qian S, Li H, et al. Predictive value of microtubule-associated protein Tau in patients with recurrent and metastatic breast cancer treated with taxane-containing palliative chemotherapy. Tumour biology: the journal of the International Society for Oncodevelopmental Biology and Medicine. May 2015; 36(5):3941-3947.

35. Koo D H, Lee H J, Ahn J H, et al. Tau and PTEN status as predictive markers for response to trastuzumab and paclitaxel in patients with HER2-positive breast cancer. Tumour biology: the journal of the International Society for Oncodevelopmental Biology and Medicine. Mar. 1 2015.

36. Wosnitzer M S, Domingo-Domenech J, Castillo-Martin M, et al. Predictive value of microtubule associated proteins tau and stathmin in patients with nonmuscle invasive bladder cancer receiving adjuvant intravesical taxane therapy. The Journal of urology. November 2011; 186(5):2094-2100.

37. Wang Q, Wang N, Shao G, et al. Relationship between gastric cancer tau protein expression and paclitaxel sensitivity. Pathology oncology research: POR. July 2013; 19(3):429-435.

39. Smoter M, Bodnar L, Grala B, et al. Tau protein as a potential predictive marker in epithelial ovarian cancer patients treated with paclitaxel/platinum first-line chemotherapy. Journal of experimental & clinical cancer research: CR. 2013; 32:25.

39. Thinnes F P. Nonmelanoma skin cancer is associated with reduced Alzheimer disease risk. Neurology. Dec. 3, 2013; 81(23):2056.

40. Yu J Q, Zhou Q, Zheng Y F, Bao Y. Expression of Vimentin and Ki-67 Proteins in Cervical Squamous Cell Carcinoma and their Relationships with Clinicopathological Features. Asian Pacific journal of cancer prevention: APJCP. 2015; 16(10):4271-4275.

41. Scholzen T, Gerdes J. The Ki-67 protein: from the known and the unknown. Journal of cellular physiology. March 2000; 182(3):311-322

42. Zhong P, Li J, Gu Y, et al. P16 and Ki-67 expression improves the diagnostic accuracy of cervical lesions but not predict persistent high risk human papillomavirus infection with CIN1. International journal of clinical and experimental pathology. 2015;8(3):2979-2986.

43. Smith T W, Lippa C F. Ki-67 immunoreactivity in Alzheimer's disease and other neurodegenerative disorders. Journal of neuropathology and experimental neurology. May 1995;54(3):297-303.

It will be seen that the advantages set forth above, and those made apparent from the foregoing description, are efficiently attained and since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention which, as a matter of language, might be said to fall there between.

What is claimed is:

1. A method of detecting neurological disease including Alzheimer's, Parkinson's, and multiple sclerosis using an oral swab and/or a skin biopsy as discussed herein.

2. A method of assessing the growth and therapeutic potential of non-melanoma skin cancers by biomarkers.

3. A method for the detection of etiology of neuro-dermatitis and delusions of parasitosis.
